(19) United States
(12) Patent Application Publication
(10) Pub. No.: US 2025/0228649 A1
(43) Pub. Date: Jul. 17, 2025

(54) METHOD AND DEVICE FOR PROVIDING BORDER LINES

(71) Applicant: OSSTEM IMPLANT CO., LTD., Seoul (KR)

(72) Inventors: Young Seok KIM, Anyang-si (KR); In Ho CHO, Seoul (KR); Kyoo Ok CHOI, Seoul (KR)

(21) Appl. No.: 18/730,755

(22) PCT Filed: Feb. 23, 2023
(86) PCT No.: PCT/KR2023/002605

§ 371 Date: Jul. 19, 2024

(30) Foreign Application Priority Data
May 10, 2022 (KR) 10-2022-0057409

(51) Int. Cl. A61C 13/00; A61C 9/00

(52) U.S. Cl. CPC A61C 13/0004; A61C 9/0053

(57) ABSTRACT

According to one embodiment, disclosed are a method, device, and recording medium for providing border lines. The method for providing border lines is characterized in that: scan data for the oral cavity is obtained; the scan data is determined on the basis of outlines illustrating the uppermost end of the upper jaw gum or the lowermost end of the lower jaw gum; border lines used for oral denture modeling are determined using the scan data; the border lines are updated on the basis of points to be updated on the border lines; and the updated border lines are displayed.

### TECHNICAL FIELD

The present disclosure relates to the technical field of determining border lines used in complete denture fabrication based on intraoral scan data.

### BACKGROUND ART

When fabricating a complete denture for an edentulous case, since there are no teeth present in scan data, lines or gum lines according to the oral structure are used. For instance, the border line, which connects arbitrary points at the uppermost or lowermost ends of the labial and buccal gums of the upper and lower jaws, can be used.

Traditionally, to generate a border line, a method of directly drawing a border line through multiple clicks or drag-and-drop inputs on the edentulous intraoral scan data has been used. However, the border lines generated using the traditional method did not match the actual border lines, and if modifications to the generated border lines were needed, direct input from the user was required, which caused inconvenience in the border line generation process and issues with low accuracy of the generated border lines.

### DISCLOSURE

### Technical Problem

An embodiment of the present disclosure aims to solve the aforementioned problems of the prior art and includes technical problems that enable the border line to be easily determined and modified using intraoral scan data when fabricating a complete denture for an edentulous jaw.

The technical problems to be solved are not limited to those mentioned above, and various other technical problems may be included within the scope apparent to a person of ordinary skill in the art.

### Technical Solution

A method for providing a border line according to a first aspect of the present disclosure may include: obtaining scan data for an oral cavity; determining a border line used for oral denture modeling on the basis of an outline representing an uppermost end of an upper jaw gum or an uppermost end of a lower jaw gum by using the scan data; updating the border line based on a point to be updated on the border line; and displaying the updated border line.

In addition, the height of an upper jaw border line in the axial direction may be lower than a height of an upper jaw outline in the axial direction, and the height of a lower jaw border line in the axial direction is higher than a height of a lower jaw outline in the axial direction.

Additionally, the height difference in the axial direction between the border line and the outline may correspond to a preset value.

Further, the border line may be positioned buccally or labially from the gum.

Further, the updating of the border line may include: displaying the border line based on an axial plane; determining the point to be updated on the basis of a first user input on the displayed border line; and updating the border line on the basis of a second user input for the point to be updated.

Further, the displaying of the border line based on the axial plane may include highlighting and displaying a section on the border line in which the height difference between the upper jaw border line and the upper jaw outline in the axial direction is smaller than a preset value.

Further, the updating of the border line on the basis of the second user input may include updating a height of the border line in the axial direction for a section adjacent to the point to be updated, on the basis of the second user input.

Further, the updating of the border line on the basis of the second user input may include updating coordinates of the border line relative to the axial plane for a section adjacent to the point to be updated, on the basis of the second user input.

Further, the method may further include determining a section adjacent to the point to be updated on the border line as a section to be updated and updating the section to be updated, based on a third user input.

Further, the updating of the border line on the basis of the second user input may include: displaying a cross-sectional image for the point to be updated; updating a height in the axial direction of the point to be updated, on the basis of the second user input applied to the cross-sectional image for the point to be updated; and updating the border line on the basis of the point to be updated.

Further, the determining of the border line by using the scan data may include, when a height difference in an axial direction between a maximum protrusion point of soft tissue located buccally or labially from the gum and the outline is smaller than a preset value, determining the border line on the basis of the maximum protrusion point.

Further, the determining of the border line by using the scan data may include: determining a border line generation point such that a height difference in an axial direction between the outline and a protrusion point of soft tissue located buccally or labially from the gum corresponds to a preset value; and determining the border line on the basis of the border line generation point, and the border line generation point may be continuously obtained from an area adjacent to the gum and continually updated.

A device for providing a border line according to a second aspect of the present disclosure may include: a receiving unit configured to obtain scan data for an oral cavity; a processor configured to use the scan data to determine a border line used for oral denture modeling on the basis of an outline representing an uppermost end of an upper jaw gum or an uppermost end of a lower jaw gum, as well as update the border line on the basis of a point to be updated on the border line; and a display configured to display the updated border line.

Further, the height of an upper jaw border line in the axial direction may be lower than a height of an upper jaw outline in the axial direction, and the height of a lower jaw border line in the axial direction is higher than a height of a lower jaw outline in the axial direction.

Further, the display may display the border line based on an axial plane, and the processor may determine the point to be updated on the basis of a first user input on the displayed border line and updating the border line on the basis of a second user input for the point to be updated.

Further, the display may display a cross-sectional image for the point to be updated, and the processor may update the height in the axial direction of the point to be updated on the basis of the second user input applied to the cross-sectional image for the point to be updated, and update the border line on the basis of the updated point to be updated.

According to a third aspect of the present disclosure, there is provided a computer-readable recording medium having stored thereon a program which, when executed by a computer system, causes the computer system to perform the method of the first aspect.

In addition, other specific details are included in the detailed description and drawings.

### Advantageous Effects

According to one embodiment, a border line can be determined based on intraoral scan data, which reduces cumbersome user operations required by conventional methods for generating the border line, thereby improving user convenience.

In addition, when modifications to the border line are necessary, only limited user operations are required, which enhances the efficiency of the modification process.

Furthermore, using the border line to fabricate a complete denture can ensure an appropriate level of retention for the complete denture, improving the satisfaction of the denture wearer.

Effects of the present disclosure are not limited to those mentioned above, and it should be understood that the effects of the present disclosure include all effects that can be inferred from the detailed description or the claims.

### DESCRIPTION OF DRAWINGS

FIG. 1 is a schematic diagram illustrating an example of the configuration of a device according to one embodiment.

FIG. 2 is a diagram illustrating an example of a method for determining border lines according to one embodiment.

FIG. 3 is a diagram for describing a method for determining an upper jaw border line and the determined upper jaw border line according to one embodiment.

FIG. 4 is a diagram illustrating an example of updating a border line according to one embodiment.

FIG. 5 is a diagram illustrating an example in which a section where the height difference between an upper jaw border line and an upper jaw outline in the axial direction is smaller than a preset value is highlighted and displayed according to one embodiment.

FIG. 6 is a flowchart illustrating each step of the operation of the device according to one embodiment.

### MODE OF THE INVENTION

While the terms used herein are selected from those that are currently most widely as possible in consideration of the functions in the present disclosure, these are subject to change depending on intents of those skilled in the art, precedents, or introduction of new technologies, etc. Further, in certain circumstances, some terms may be arbitrarily chosen by the Applicant, in which case the corresponding meaning will be defined in detail in the corresponding part of the description. Accordingly, the terms used herein should be defined based on the meanings thereof and the overall content of the disclosure, rather than simply based on what these terms are called.

Throughout the specification, it will be understood that when a component “includes (or comprises)” an element, unless there is another opposite description thereto, it should be understood that the component does not exclude another element but may further include another element. Furthermore, the term “unit (or part)” used in the specification refers to a unit for processing at least one function or operation, and this may be realized in the form of hardware, software, or in a combination of both hardware and software.

Embodiments of the present invention will now be described in detail with reference to the accompanying drawings so that those skilled in the art can easily practice the embodiments of the present disclosure. However, the present disclosure may be implemented in many different forms and is not limited to the embodiments described herein.

Hereinafter, a plurality of embodiments will be described in detail with reference to the drawings.

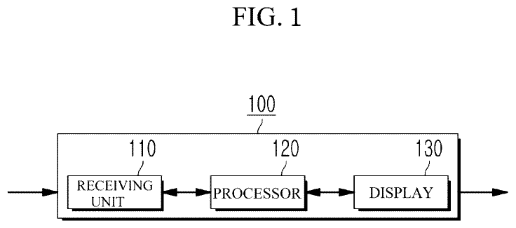

FIG. 1 is a schematic diagram illustrating an example of the configuration of a device 100 according to one embodiment.

Referring to FIG. 1, a device 100 may include a receiving unit 110, a processor 120, and a display 130.

The receiving unit 110 may obtain scan data (e.g., 3-dimensional (3D) scan image, etc.) of an oral cavity from a capturing device (not shown). Here, the scan data may include scan data of the upper and low jaws in an edentulous state, and scan data of edentulous upper and lower jaw impressions or models. Since intraoral scan data comprehensively refers to scan data of the oral cavity, it may include impression scan data.

Additionally, the receiving unit 110 may receive information provided from an external device (not shown). For example, the receiving unit 110 may include a wired or wireless communication device capable of receiving various types of information by being connected to the external device or external component (not shown) through a network or a signal processing module.

The processor 120 according to one embodiment may use the scan data to determine a border line used for oral denture modeling on the basis of the outline representing the uppermost end of the upper jaw gum or the lowermost end of the lower jaw gum.

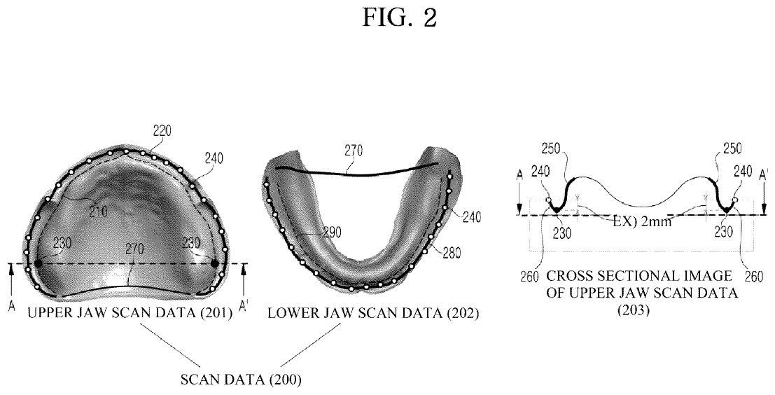

Here, referring to a cross-sectional image 203 of the upper jaw scan data in FIG. 2, the uppermost end 230 of the upper jaw gum may be obtained on the basis of the point where the curvature change of the bend formed between a buccal gum surface 250 and the opposing buccal skin surface 260 reaches a maximum value (the point where the buccal gum surface 250 and the buccal skin surface 260 are connected in the image). One or more uppermost ends 230 of the upper jaw gum may be obtained from the upper jaw scan data 201 or the cross-sectional image 203 of the upper jaw scan data.

Similarly, the lowermost end of the lower jaw gum may be obtained on the basis of the point where the curvature change of the bend formed between the buccal gum surface of the lower jaw and the opposing buccal skin surface of the lower jaw reaches a maximum value. One or more lowermost end of the lower jaw gum may be obtained from lower jaw scan data 202 or a cross-sectional image of the lower jaw scan data.

In addition, the uppermost end of the upper jaw gum and the lowermost end of the lower jaw gum on the labial side may also be obtained in the same manner as described above.

The upper jaw outline 210 may be obtained on the basis of a line connecting the uppermost ends 230 of the upper jaw gum, and the lower jaw outline 290 may be obtained on the basis of a line connecting the lowermost ends of the lower jaw gum. Further, the term “outline” 210 or 290 may be used to include both the upper jaw outline 210 and the lower jaw outline 290.

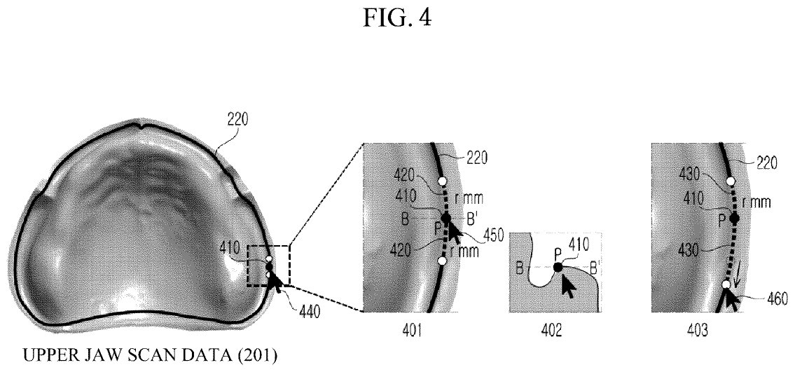

The processor 120 may update the border line 220 or 280 on the basis of a point to be updated on the border line 220 or 280 which includes the upper jaw border line 220 and the lower jaw border line 280. Accordingly, the display 130 may display the updated border line (refer to 403 in FIG. 4).

In one embodiment, the height of the upper jaw border line 220 in the axial (horizontal) direction may be lower than the height of the upper jaw outline 210 in the axial direction, and the height of the lower jaw border line 280 in the axial direction may be higher than the height of the lower jaw outline 290 in the axial direction. The height differences in the axial direction between the upper and lower jaw border lines 220 and 280 and the upper and lower jaw outlines 210 and 290 may correspond to a preset value (e.g., 1.5 mm, 2 mm, 2.5 mm, etc.).

In one embodiment, the processor 120 may determine the point to be updated on the basis of a first user input on the displayed border line 220 or 280 relative to the axial plane, and update the border line 220 or 280 on the basis of a second user input for the point to be updated. The axial plane may be interpreted to include various planes corresponding to the horizontal plane. For example, the axial plane may be interpreted to include the occlusal plane.

Further, when a cross-sectional image for the point to be updated is displayed, the processor 120 may update the height in the axial direction of the point to be updated, on the basis of the second user input applied to the cross-sectional image for the point to be updated, and update the border line 220 or 280 on the basis of the updated point.

Furthermore, the processor 120 may perform a series of operations to determine and update the border lines 220 and 280, and is electrically connected to the receiving unit 110, the display 130, and other components not shown to control the data flow between them. For this purpose, the processor 120 may be implemented as a central processing unit (CPU) that controls the overall operation of the device 100.

In addition, it will be understood by those skilled in the art that, in addition to the components shown in FIG. 1, other general components may be further included in the device 100. According to one embodiment, the device 100 may further include an algorithm for processing the scan data 200, a user interface unit for receiving user input, and a storage unit configured to store the data described throughout the specification.

The embodiments described above with reference to FIG. 1 will be described in more detail with reference to FIGS. 2 to 6.

FIG. 2 is a diagram illustrating an example of a method for determining border lines 220 and 280 according to one embodiment.

Referring to FIG. 2, the device 100 may determine the upper jaw outline 210 obtained on the basis of a line connecting one or more uppermost ends 230 of the upper jaw gum, and the lower jaw outline 290 obtained on the basis of a line connecting one or more lowermost ends of the lower jaw gum.

The outlines 210 and 290 described in this disclosure generally refer to lines obtained based on the “oral vestibule,” but they are not necessarily limited thereto.

The upper jaw border line 220 according to one embodiment may be determined on the basis of border line generation points 240, which are points horizontally spaced in the direction of the buccal skin by a preset value (e.g., 1.5 mm, 2 mm, 2.5 mm, etc.) from the uppermost end 230 of the upper jaw gum in the upper jaw scan data 201. The number of border line generation points 240 may correspond to the number of uppermost ends 230 of the upper jaw gum, and the border lines 220 and 280 may be obtained on the basis of lines connecting one or more border line generation points 240.

In another embodiment, the upper jaw border line 220 may be determined on the basis of border line generation points 240, which are points vertically spaced in the direction of the buccal skin by a preset interval from the uppermost end 230 of the upper jaw gum in the cross-sectional image 203 of the upper jaw scan data. The cross-sectional image 203 of the upper jaw scan data may be obtained based on A-A′ line, which is a line connecting two points A and A′ of the upper jaw scan data 201. For example, the cross-sectional image 203 of the upper jaw scan data may be a cross-sectional image that is obtained when the upper jaw scan data 201 is taken along the A-A′ line. In one embodiment, the line A-A′ connecting two points of the upper jaw scan data 201 may be located at the uppermost ends 230 of the upper jaw gum.

A detailed description of the border line generation points 240 will be provided below with reference to FIG. 4.

The lower jaw border line 280 may also be obtained in the same manner as the upper jaw border line 220. Additionally, the same method applied to the labial and buccal sides of the upper and lower jaws may be applied to the lingual side of the mandible for the lower jaw border line 280.

In one embodiment, the device 100 may obtain a user-defined line 270 representing the posterior points of the upper and lower jaw oral cavities on the basis of user input (e.g., drag-and-drop input) applied to the upper jaw scan data 201 and lower jaw scan data 202. The user-defined line 270 may be a line to connect the border lines 220 and 280, and the border lines 220 and 280 may be determined to include the user-defined line 270.

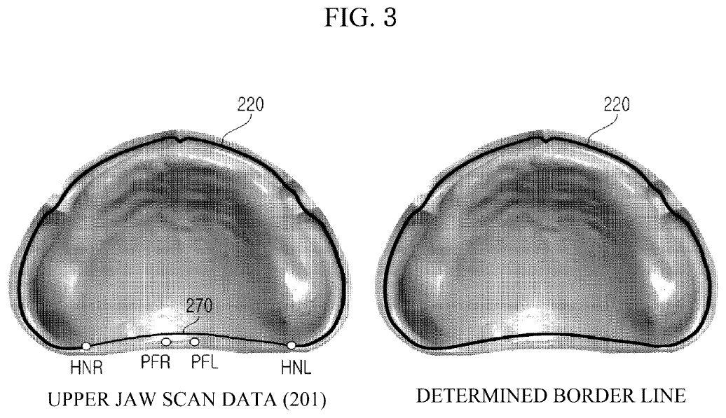

FIG. 3 is a diagram for describing the method for determining the upper jaw border line 220 and the determined upper jaw border line 220 according to one embodiment.

Referring to FIG. 3, the device 100 according to one embodiment may connect both end points of the user-defined line 270 to both end points of the upper jaw border line 220 based on preset landmarks, hamular notch right (HNR) and hamular notch left (HNL), obtained from the upper jaw scan data 201. Then, after moving the other preset landmarks, palatine foveae right (PFR) and palatine foveae left (PFL), forward by a preset value (e.g., 1.5 mm, 2 mm, 2.5 mm, etc.), the device 100 may adjust the user-defined line 270 using HNR, HNL, PFR, and PFL, and connect the user-defined line 270 and the upper jaw border line 220 to determine the final upper jaw border line 220 as shown in FIG. 3. HNR, HNL, PFR, and PFL are types of landmarks: HNR refers to the right hamular notch, HNL to the left hamular notch, PFR to the right palatine fovea, and PFL to the left palatine fovea.

In one embodiment, if the user-defined line 270 is not obtained, the device 100 may determine the final upper jaw border line 220 by moving PFR and PFL forward by a preset value and then connecting PFR, PFL, HNR, HNL, and the upper jaw border line 220.

In one embodiment, the height of the upper jaw border line 220 in the axial direction may be lower than the height of the upper jaw outline 210, and the height of the lower jaw border line 280 may be higher than the height of the lower jaw outline 290.

For example, in the axial direction, the upper jaw border line 220 may be located at a relatively lower point than the upper jaw outline 210. In this case, the height difference between the upper jaw border line 220 and the upper jaw outline 210 may correspond to a preset value (e.g., 1.5 mm, 2 mm, 2.5 mm, etc.).

Additionally, in the axial direction, the lower jaw border line 280 may be located at a relatively higher point than the lower jaw outline 290. In this case, the height difference between the lower jaw border line 280 and the lower jaw outline 290 may correspond to the preset value.

Furthermore, the border lines 220 and 280 may be positioned buccally or labially from the gums.

FIG. 4 is a diagram illustrating an example of updating the border line 220 or 280 according to one embodiment.

Referring to FIG. 4, the device 100 may display the upper jaw border line 220 relative to the axial plane, and determine a point 410 to be updated on the basis of a first user input 440 applied to the displayed upper jaw border line 220. Additionally, the device 100 may update the upper jaw border line 220 on the basis of a second user input 450 applied to the point 410 to be updated.

For example, as shown in the upper jaw scan data 201 of FIG. 4, the device 100 may determine a point on the upper jaw border line 220, which is determined according to the first user input 440, as the point 410 to be updated. As shown in the enlarged image 401 of the point to be updated, the device 100 may determine a section adjacent to the point 410 to be updated by a preset value as a section 420 to be updated. When the second user input 450 is applied to the point 410 to be updated, the device 100 may update the height in the axial direction or the coordinates relative to the axial plane of the section 420 to be updated, on the basis of the second user input 450. For example, the cross-sectional image 402 of the point to be updated may be obtained based on B-B′ line, which is a line connecting two points B and B′ in the enlarged image 401 of the point to be updated. For example, the cross-sectional image 402 of the point to be updated may be a cross-sectional image that is obtained when the enlarged image 401 of the point to be updated is taken along the B-B′ line. If the second user input 450 is additionally applied to the point 410 to be updated which is displayed in the cross-sectional image 402 of the point to be updated, the device 100 may update the position of the point 410 to be updated.

Specifically, referring to FIG. 4 to explain an embodiment of updating the upper jaw border line 220, if the point 410 to be updated on the upper jaw border line 220 displayed on the upper jaw scan data 201 or the cross-sectional image of the point to be updated is determined, the device 100 may determine a section adjacent to the point 410 to be updated on the upper border line 220 as a section 420 to be updated. The section 420 to be updated may represent a target section to which an update input is applied corresponding to the second user input 450. Additionally, if the device 100 obtains a third user input 460 for the section 420 to be updated, the device 100 may adjust the range of the section 420 to be updated to correspond to the third user input 460. As shown in an image 403 in FIG. 4 illustrating that the section to be updated is updated, the range of the section 420 to be updated may be widened or narrowed to correspond to the third user input 460, and the upper jaw border line 220 may be updated based on the updated section 430. This embodiment may also be performed on the scan data 200 or modeling data that includes the point 410 to be updated.

Thereafter, the device 100 may update the height in the axial direction or the coordinates relative to the axial plane of the section 420 to be updated based on the obtained second user input 450.

In one embodiment, when the point 410 to be updated is determined on the basis of the first user input 440, the device 100 may display an image of a preset area corresponding to the point 410 to be updated as shown in the enlarged image 401 of the point to be updated.

If the second user input 450 is applied to the enlarged image 401 of the point to be updated, the device 100 may update the height of the point 410 to be updated in the axial direction. Thereafter, the device 100 may update the upper jaw border line 220 on the basis of the updated point 410 to be updated.

In one embodiment, when the height difference in the axial direction between the maximum protrusion point of the soft tissue located buccally or labially from the gum and the outlines 210 and 290 is smaller than a preset value, the device 100 may determine the upper jaw border line 220 on the basis of the maximum protrusion point.

Here, the term “soft tissue” may refer to, for example, referring to FIG. 2, the soft skin tissue facing the buccal gum surface 250, shown in the cross-sectional image 203 of the upper jaw scan data, and labial gum surface. It also includes the buccal skin surface 260 and labial skin surface, as well as the soft skin tissue present in the directions of the buccal and labial skin surfaces. For example, soft tissue may refer to the soft skin tissue in the oral cavity that faces the gums and gum surfaces but excludes the gums and gum surfaces.

Additionally, referring to the cross-sectional image 203 of the upper jaw scan data in FIG. 2, the maximum protrusion point may be defined as a point among protrusion points of the soft tissue that protrude perpendicular to the axial direction, where a protrusion value is greater than or equal to a preset value based on a point on the upper jaw outline 210 that faces the protrusion points. Accordingly, in one example, the maximum protrusion point may include the border line generation point 240. Specifically, the border line generation point 240 may be a point that is vertically or horizontally spaced by a preset interval in the direction of the buccal or labial skin from the upper jaw outline 210.

The soft tissue and maximum protrusion points for the upper jaw have the same meaning for the lower jaw, and the soft tissue and maximum protrusion points for the lower jaw may be obtained in the same manner as those for the upper jaw.

In one embodiment, if the height difference in the axial direction between the maximum protrusion points and the outlines 210 and 290 is smaller than the preset value, the border lines 220 and 280 may not be clearly defined, or they may be unsuitable for wearing a complete denture, potentially reducing the retention of the fabricated complete denture. Therefore, the device 100 may determine the border lines 220 and 280 on the basis of the maximum protrusion points if the height difference in the axial direction between the maximum protrusion points and the outline 210 and 290 is smaller than the preset value.

In another embodiment, if the height difference in the axial direction between the maximum protrusion points and the outlines 210 and 290 is greater than the preset value, the device 100 may determine the border lines 220 and 280 on the basis of the points corresponding to the height difference in the axial direction by the preset value from the outlines 210 and 290.

In one embodiment, the device 100 may determine the border line generation points 240 such that the height difference in the axial direction between the outlines 210 and 290 and the protrusion points of the soft tissue located buccally or labially from the gums corresponds to the preset value. The device 100 may determine the border lines 220 and 280 on the basis of the determined border line generation points 240. The border line generation points 240 may be continuously obtained from the areas adjacent to the gums and continually updated (updated in real time?).

The border line generation points 240 may be continually updated, for example, when the point 410 to be updated on the upper jaw border line 220 is modified on the basis of the second user input 450, and they may be continually determined and updated such that the height difference in the axial direction between the protrusion points of the soft tissue located buccally or labially from the gums and the outlines 210 and 290 on the scan data 200 corresponds to the preset value.

Additionally, in the same manner as the embodiment for the upper jaw, the border line generation points 240 for the lower jaw may be determined, and the lower jaw border line 280 may be determined on the basis of the determined border line generation points 240.

When an update to the border lines 220 and 280 is necessary, the update can be easily performed with simple user operations without going through complex steps, thereby improving the efficiency of updating the border lines 220 and 280 and the complete denture fabrication process using them.

In one embodiment, the device 100 may store and manage the information regarding the updated border line generation points (e.g., coordinates relative to the axial plane) according to the previous embodiments and use the information regarding the updated border line generation points to determine new border lines in the future.

If the similarity between each scan data is greater than or equal to a preset value, the device 100 may determine a new border line or new border line generation points according to new scan data using the information regarding the previously updated border line generation points (or the updated border line) obtained on the basis of the scan data 200. Here, a preset algorithm may be applied to the method of determining the new border line or new border line generation points based on the information regarding the previously updated border line generation points. This algorithm may determine whether the previously updated border line generation points match the new scan data with less than an acceptable value.

In another embodiment, if the similarity between each scan data is greater than or equal to a preset value, the device 100 may display the information regarding the previously updated border line generation points on the new scan data and may allow the user to refer to the displayed information to determine the new border line or new border line generation points.

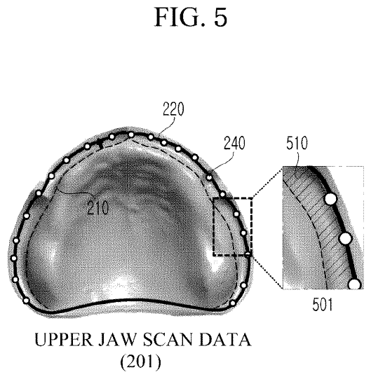

Accordingly, the user may relatively easily determine the new border line using the previously generated border lines 220 and 280 and border line generation points 240. FIG. 5 is a diagram illustrating an example in which a section 510 where the height difference between the upper jaw border line 220 and an upper jaw outline 210 in the axial direction is smaller than a preset value is highlighted and displayed according to one embodiment.

Referring to FIG. 5, the device 110 may determine a section 510 where the height difference in the axial direction between the upper jaw border line 220 and the upper jaw outline 210 is smaller than a preset value.

For example, the device 100 may determine the height difference in the axial direction on the basis of the upper jaw scan data 201 and display the section 510 in which the determined height difference is smaller than the preset value to highlight it, as shown in FIG. 5.

By separately highlighting and displaying the section 510 in which the height difference in the axial direction between the upper jaw border line 220 and the upper jaw outline 210 is smaller than the preset value, as shown in FIG. 5, the device 100 may allow the user to easily identify the section 510 in which the height difference in the axial direction between the upper jaw border line 220 and the upper jaw outline 210 is smaller than the preset value, and may also help the user to easily recognize that the upper jaw border line 220 in the section 510 in which the height difference in the axial direction between the upper jaw border line 220 and the upper jaw outline 210 is smaller than the preset value has been determined on the basis of the maximum protrusion points of the soft tissue.

There may be several methods for the device 100 to highlight and display the section 510 in which the height difference is smaller than the preset value. For example, the device 100 may display the section 510 with a smaller height difference in a different color (e.g., red). In another example, the device 100 may provide an enlarged additional screen of the section 510 with a smaller height difference. In addition, by highlighting and displaying the section 510 where the height difference is smaller than the preset value in various ways, the device 100 may allow the user to easily recognize whether the border line 220 or 280 determined for that section needs modification.

The embodiments described above can be easily understood to apply equally to the lower jaw based on all the embodiments described in this disclosure.

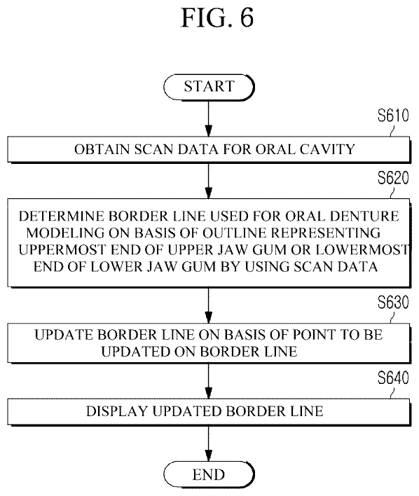

FIG. 6 is a flowchart illustrating each step of the operation of the device 100 according to one embodiment.

Referring to FIG. 6, in step S610, the device 100 may acquire scan data 200 of an oral cavity. The scan data 200 may include scan data 200 of the upper and lower jaws in an edentulous state. Additionally, the device 100 may further receive impression images of the upper or lower jaw, oral models, a cross-sectional image 203 of the upper jaw scan data, and a cross-sectional image of the lower jaw scan data through the receiving unit 110, and use this information to determine the border lines 220 and 280.

In step S620, the device 100 may determine the border line 220 or 280 used for oral denture modeling on the basis of the outline 210 or 290 representing the uppermost end of the upper jaw gum or the lowermost end of the lower jaw gum using the scan data 200.

For example, the border lines 220 and 280 may be determined on the basis of the upper jaw border line 220 and the lower jaw border line 280 that are formed by connecting the border line generation points 240 of the upper and lower jaws obtained on the basis of the upper jaw scan data 201 or the cross-sectional image 203 of the upper jaw scan data and the lower jaw scan data 202 or the cross-sectional image of the lower jaw scan data.

In one embodiment, the device 100 may display the obtained user-defined line 270 and the upper jaw border line 220 on the upper jaw scan data 201, connect both end points of the user-defined line 270 to both end points of the upper jaw border line 220 based on preset landmarks, HNR) and HNL, obtained from the upper jaw scan data 201, then, after moving the other preset landmarks, PFR and PFL, forward by a preset value (e.g., 1.5 mm, 2 mm, 2.5 mm, etc.), adjust the user-defined line 270 using HNR, HNL, PFR, and PFL. Then, the device 100 may connect the user-defined line 270 and the upper jaw border line 220 to determine the final upper jaw border line 220.

In one embodiment, if the user-defined line 270 is not obtained, the device 100 may determine the final upper jaw border line 220 by moving PFR and PFL forward by a preset value and then connecting PFR, PFL, HNR, HNL, and the upper jaw border line 220.

In step S630, the device 100 may update the upper jaw border line 220 on the basis of the point 410 to be updated on the upper jaw border line 220. Additionally, the device 100 may update the lower jaw border line 280 on the basis of the point to be updated on the lower jaw border line 280.

For example, referring to FIG. 4 to explain the embodiment of updating the upper jaw border line 220, if the point 410 to be updated on the upper jaw border line 220 is determined, the device 100 may determine a section adjacent to the point 410 on the upper jaw border line 220 to be updated as a section 420 to be updated. The section 420 to be updated may represent a target section to which an update input is applied corresponding to the second user input 450. Additionally, if the device 100 obtains a third user input 460 for the section 420 to be updated, the device 100 may adjust the range of the section 420 to be updated to correspond to the third user input 460. As shown in an image 403 in FIG. 4 illustrating that the section to be updated is updated, the range of the section 420 to be updated may be widened or narrowed to correspond to the third user input 460, and the upper jaw border line 220 may be updated based on the updated section 430.

Thereafter, the device 100 may update the height in the axial direction or the coordinates relative to the axial plane of the section 420 to be updated based on the obtained second user input 450.

In step S640, the device 100 may display the updated border lines.

When the updated border lines are displayed, the user may use the updated border lines in the fabrication of complete dentures, which reduces the cumbersome user operations required for generating border lines 220 and 280 according to conventional methods, thereby improving user convenience. Additionally, using the border lines 220 and 280 or the updated border lines to fabricate complete dentures may ensure an appropriate level of retention for the complete dentures, improving the satisfaction of the denture wearer.

In one embodiment, the device 100 may receive user input corresponding to a request for providing a guide border line. When receiving the user input corresponding to the request for providing a guide border line, the device 100 may provide at least one guide border line.

For example, the device 100 may obtain coordinate values (hereafter, first coordinate values) of each border line generation point 240 relative to the axial plane on the basis of the displayed scan data 200.

Additionally, the device 100 may obtain coordinate values (hereafter, second coordinate values) of each border line generation point 240 relative to the axial plane on the basis of pre-stored scan data 200.

The device 100 may compare a plurality of first coordinate values with a plurality of second coordinate values, and based on the comparison results, determine a close second coordinate value that is at least one second coordinate value whose difference from the first coordinate values is less than or equal to a preset value.

The device 100 may determine at least one pre-stored border line 220 or 280 that includes at least a predetermined number of border line generation points 240 corresponding to the close second coordinate value as the guide border line, and provide the determined guide border line when the user input corresponding to the request for providing a guide border line is received.

As described above, the device 100 may support the user's smooth creation of the border line 220 or 280 by providing at least one pre-stored border line 220 or 280 as a guide border line that includes border line generation points 240 located at similar or identical positions as the border line generation points 240 obtained on the basis of the currently displayed scan data 200.

In another embodiment, the device 100 may preferentially provide at least one guide border line from a plurality of pre-stored guide border lines.

For example, the device 100 may determine the priority for providing at least one guide border line based on the weight given in order of the number of border line generation points 240 of the close second coordinate value whose difference from the first coordinate value is less than or equal to the preset value, the expected number of second user inputs 450, and the height difference in the axial direction between the maximum protrusion point and the outline 210 or 290, and it may preferentially provide at least one guide border line based on the determined priority.

For example, the device 100 may preferentially provide at least one guide border line that includes a preset number or more of border line generation points 240 corresponding to the close second coordinate value whose difference from the multiple first coordinate values is less than or equal to the preset value. By doing so, the device 100 may preferentially provide the border line 220 or 280 that is expected to be most suitable for the oral cavity targeted for the generation of the border line 220 or 280. Thus, the working time for generating the border line 220 or 280 can be significantly reduced, and this can also lead to high user satisfaction when fabricating the complete denture based on it, thereby improving the efficiency of the work.

Additionally, the expected number of second user inputs may represent the number of second user inputs 450 anticipated to be necessary if a plurality of guide border lines are displayed overlapped on the currently displayed scan data 200. A guide border line with a lower expected number of second user inputs, less than or equal to a preset number, may be considered suitable for the currently displayed scan data 200, so the number of user update inputs can be reduced, allowing for more efficient and quicker work. Therefore, the expected number of second user inputs may be given the second highest weight.

Moreover, if the height difference in the axial direction between the maximum protrusion point of a target patient and the outline 210 or 290 is smaller than a preset value, the border line 220 or 280 determined on the basis of the scan data 200 that includes a height difference value that is the same or similar to the corresponding height difference of the target patient may be provided as a guide border line, making it possible to quickly provide the border line 220 or 280 suitable for a patient with unusual oral structures. However, since the number of patients with such unusual oral structures is relatively small compared to other typical patients, the third highest weight may be assigned to the height difference in the axial direction between the maximum protrusion point and the outline 210 or 290.

In another embodiment, additional weights may be assigned as fourth, fifth, and sixth priorities to the similarity in the proportion of oral cavity size based on the scan data 200, gender, and expected pressure on each tooth position according to mastication movements, respectively.

Based on these differently assigned weights, the device 100 may determine the priority for preferentially providing at least one guide border line from the plurality of pre-stored guide border lines. By providing at least one guide border line based on the determined priority, the user's work efficiency and working time may be improved.

In another embodiment, if the height difference in the axial direction between the maximum protrusion point of the target patient and the outline 210 or 290 is smaller than a predetermined value, the device 100 may determine the priority for providing at least one guide border line from a plurality of guide border lines based on the weights assigned in the order of the number of border line generation points 240 corresponding to the close second coordinate value, the similarity in the height difference value in the axial direction between the maximum protrusion point and the outline 210 or 290, the expected number of second user inputs, and the similarity in the proportion of the oral cavity size based on the scan data 200, and the device 100 may provide at least one guide border line based on the determined priority.

Additionally, if the height difference in the axial direction between the maximum protrusion point of the target patient and the outline 210 or 290 is smaller than a predetermined value, the device 100 may determine the border line generation points 240 on the border line 220 or 280 determined on the basis of the maximum protrusion point.

For example, in the case of an oral cavity where the height difference in the axial direction between the maximum protrusion point and the outline 210 or 290 is smaller than a preset value, it can be considered that at least one guide border line 220 or 280, including a preset number or more of border line generation points 240 corresponding to one or more close second coordinate values, has high suitability for fabricating a complete denture. Therefore, the highest weight may be given to the number of border line generation points 240 that include one or more close second coordinate values.

In addition, if the height difference in the axial direction between the maximum protrusion point of the target patient and the outline 210 or 290 is smaller than the preset value, the device 100 determine the border line 220 or 280 on the basis of the maximum protrusion point. In such cases where the height difference in the axial direction between the maximum protrusion point of the target patient and the outline 210 or 290 is smaller than the preset value, the guide border line with similar height difference values between the maximum protrusion points and the outline 210 or 290 obtained from each preset point in the displayed scan data 200 can be considered suitable for the displayed scan data 200. Therefore, the device 100 may assign the second highest weight to the similarity in the height difference value in the axial direction between the maximum protrusion point and the outline 210 or 290.

Additionally, a guide border line with a lower expected number of second user inputs, less than or equal to a preset number, may be considered suitable for the currently displayed scan data 200. Hence, the third highest weight may be assigned to the expected number of second user inputs.

Furthermore, if the height difference in the axial direction between the maximum protrusion point of the target patient and the outline 210 or 290 is smaller than the preset value, the size of the oral cavity is also expected to differ from the general size of an oral cavity. Therefore, it is preferable to preferentially provide a guide border line based on the scan data 200 with a similarity in the proportion of the oral cavity size based on the displayed scan data 200, greater than or equal to the preset value. Hence, the fourth highest weight may be assigned to the similarity in the ratio of the oral cavity size based on the scan data 200.

Based on the differently assigned weights, the device 100 may determine the priority for providing at least one guide border line among the plurality of pre-stored guide border lines. By providing at least one guide border line based on the determined priority, the border line 220 or 280 for an oral cavity where the height difference in the axial direction between the maximum protrusion point and the outline 210 or 290 is smaller than the preset value may be obtained relatively easily.

In the aforementioned embodiments, the weight assigned to the number of border line generation points 240 with the same coordinates based on the axial plane may be determined to be proportional to the number of border line generation points 240 with the same coordinates based on the axial plane. The weight assigned to the expected number of second user inputs may be determined to be inversely proportional to the expected number of second user inputs. The weight assigned to the height difference in the axial direction between the maximum protrusion point and the outline 210 or 290 may be determined to be proportional to the similarity in the height difference value in the axial direction between the maximum protrusion point and the outline 210 or 290. The weight assigned to the similarity in the proportion of the oral cavity size based on the scan data 200 may be determined to be proportional to the similarity in the proportion of the oral cavity size based on the scan data 200.

Additionally, the embodiments related to the weights disclosed in FIG. 6 are exemplary embodiments and should not be interpreted as being limited to the described embodiments.

It should be appreciated that the order and combination of the steps shown above is merely an embodiment of the present disclosure, and the order, combination, branch, function and the performing subject may vary to be implemented with addition, fewer, or different steps without departing from the essential characteristics of each component described in the specification. Throughout this specification, the term “provide (or providing)” may be interpreted as comprehensively including a process in which an object obtains specific information or directly or indirectly transmits or receives specific information to or from a specific object and including the performance of related operations required in this process.

Various embodiments set forth herein may be implemented as software comprising one or more instructions stored in a storage medium (e.g., memory) that is readable by a machine (e.g., a display device or a computer). For example, a processor (e.g., the processor 120) of the machine may invoke at least one of the one or more instructions stored in the storage medium, and execute it. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

It will be understood by those of ordinary skill in the art that various changes in form and details may be made therein without departing from the characteristics described above. The disclosed methods should be considered in a descriptive sense only and not for purposes of limitation. The scope of the present disclosure is defined by the appended claims rather than by the foregoing description, and all differences within the scope of equivalents thereof should be construed as being included in the present disclosure.

1. A method for providing border lines, comprising:
obtaining scan data for an oral cavity;
determining a border line used for oral denture modeling on the basis of an outline representing an uppermost end of an upper jaw gum or an uppermost end of a lower jaw gum by using the scan data;
updating the border line based on a point to be updated on the border line; and
displaying the updated border line.

2. The method of claim 1, wherein a height of an upper jaw border line in an axial direction is lower than a height of an upper jaw outline in the axial direction, and a height of the lower jaw border line in the axial direction is higher than a height of a lower jaw outline in the axial direction.

3. The method of claim 1, wherein a height difference in an axial direction between the border line and the outline corresponds to a preset value.

4. The method of claim 1, wherein the border line is positioned buccally or labially from the gum.

5. The method of claim 1, wherein the updating of the border line comprises:
displaying the border line based on an axial plane;
determining the point to be updated on the basis of a first user input on the displayed border line; and
updating the border line on the basis of a second user input for the point to be updated.

6. The method of claim 5, wherein the displaying the border line based on the axial plane comprises highlighting and displaying a section on the border line in which the height difference between an upper jaw border line and an upper jaw outline in the axial direction is smaller than a preset value.

7. The method of claim 5, wherein the updating of the border line on the basis of the second user input comprises updating a height of the border line in the axial direction for a section adjacent to the point to be updated, on the basis of the second user input.

8. The method of claim 5, wherein the updating of the border line on the basis of the second user input comprises updating coordinates of the border line relative to the axial plane for a section adjacent to the point to be updated, on the basis of the second user input.

9. The method of claim 5, further comprising:
determining a section adjacent to the point to be updated on the border line as a section to be updated; and
updating the section to be updated, based on a third user input.

10. The method of claim 5, wherein the updating of the border line on the basis of the second user input comprises:
displaying a cross-sectional image for the point to be updated;
updating a height in the axial direction of the point to be updated, on the basis of the second user input applied to the cross-sectional image for the point to be updated; and
updating the border line on the basis of the point to be updated.

11. The method of claim 1, wherein the determining of the border line by using the scan data comprises, when a height difference in an axial direction between a maximum protrusion point of soft tissue located buccally or labially from the gum and the outline is smaller than a preset value, determining the border line on the basis of the maximum protrusion point.

12. The method of claim 1, wherein the determining of the border line by using the scan data comprises:
determining a border line generation point such that a height difference in an axial direction between the outline and a protrusion point of soft tissue located buccally or labially from the gum corresponds to a preset value; and
determining the border line on the basis of the border line generation point, and
the border line generation point is continuously obtained from an area adjacent to the gum and continually updated.

13. A device for providing a border line, comprising:
a receiving unit configured to obtain scan data for an oral cavity;
a processor configured to use the scan data to determine a border line used for oral denture modeling on the basis of an outline representing an uppermost end of an upper jaw gum or an uppermost end of a lower jaw gum, as well as
update the border line on the basis of a point to be updated on the border line; and
a display configured to display the updated border line.

14. A computer-readable recording medium having stored thereon a program which, when executed by a computer system, causes the computer system to perform a method for providing border lines, wherein the method comprises:
obtaining scan data for an oral cavity;
determining a border line used for oral denture modeling on the basis of an outline representing an uppermost end of an upper jaw gum or an uppermost end of a lower jaw gum by using the scan data;
updating the border line based on a point to be updated on the border line; and
displaying the updated border line.

15. The computer-readable recording medium of claim 14, wherein a height of an upper jaw border line in an axial direction is lower than a height of an upper jaw outline in the axial direction, and a height of the lower jaw border line in the axial direction is higher than a height of a lower jaw outline in the axial direction.

16. The computer-readable recording medium of claim 14, wherein a height difference in an axial direction between the border line and the outline corresponds to a preset value.

17. The computer-readable recording medium of claim 14, wherein the border line is positioned buccally or labially from the gum.

18. The computer-readable recording medium of claim 14, wherein the updating of the border line comprises:
displaying the border line based on an axial plane;
determining the point to be updated on the basis of a first user input on the displayed border line; and
updating the border line on the basis of a second user input for the point to be updated,
wherein the displaying the border line based on the axial plane comprises highlighting and displaying a section on the border line in which the height difference between an upper jaw border line and an upper jaw outline in the axial direction is smaller than a preset value,
wherein the updating of the border line on the basis of the second user input comprises updating a height of the border line in the axial direction for a section adjacent to the point to be updated, on the basis of the second user input,
wherein the updating of the border line on the basis of the second user input comprises updating coordinates of the border line relative to the axial plane for a section adjacent to the point to be updated, on the basis of the second user input,
wherein the method further comprises determining a section adjacent to the point to be updated on the border line as a section to be updated and updating the section to be updated, based on a third user input, and
wherein the updating of the border line on the basis of the second user input comprises:
displaying a cross-sectional image for the point to be updated;
updating a height in the axial direction of the point to be updated, on the basis of the second user input applied to the cross-sectional image for the point to be updated; and
updating the border line on the basis of the point to be updated.

19. The computer-readable recording medium of claim 14, wherein wherein the determining of the border line by using the scan data comprises, when a height difference in an axial direction between a maximum protrusion point of soft tissue located buccally or labially from the gum and the outline is smaller than a preset value, determining the border line on the basis of the maximum protrusion point.

20. The computer-readable recording medium of claim 14, wherein the determining of the border line by using the scan data comprises:
determining a border line generation point such that a height difference in an axial direction between the outline and a protrusion point of soft tissue located buccally or labially from the gum corresponds to a preset value; and
determining the border line on the basis of the border line generation point, and
the border line generation point is continuously obtained from an area adjacent to the gum and continually updated.
